(19) United States
(12) Patent Application Publication
(10) Pub. No.: US 2026/0260339 A1
(43) Pub. Date: Sep. 3, 2026

(54) PROCESSING METHOD OF AN ULTRASOUND IMAGE TO ASSOCIATE INFORMATION SUPPORTING TUMOUR DIAGNOSIS OF CYSTS AND SOLID LESIONS

(71) Applicant: SYNDIAG S.R.L., Torino (IT)

(72) Inventors: Rosilari BELLACOSA MAROTTI, Torino (IT); Federica GERACE, Torino (IT); Daniele CONTI, Torino (IT); Flavia DE SIMONE, Torino (IT); Martina LILLA, Torino (IT); Pio Raffaele FINA, Torino (IT)

(73) Assignee: SYNDIAG S.R.L., Torino (IT)

(21) Appl. No.: 18/875,201

(22) PCT Filed: Jun. 15, 2023
(86) PCT No.: PCT/IB2023/056227

§ 371 Date: Dec. 16, 2024

(30) Foreign Application Priority Data
Jun. 15, 2022 (IT) 102022000012623

(51) Int. Cl. G06T 7/00; G06T 7/12; G06T 7/194; G06V 10/25; G06V 10/764

(52) U.S. Cl. CPC G06T 7/0012; G06T 7/12; G06T 7/194; G06V 10/25; G06V 10/764; G06T 2207/10132; G06T 2207/20036; G06T 2207/20081; G06T 2207/30096; G06V 2201/031

(57) ABSTRACT

A computerized method of processing a representative ultrasound image of a solid ovarian, renal, pancreatic, bladder or cyst lesion e.g. ovarian, renal, hepatic, pancreatic, bladder, gallbladder subject to a subsequent medical diagnosis, including the phases of: segmenting the image using a first algorithm to: identify at least one outline and at least one corresponding object delimited by the outline and representative of a biological tissue of a cyst or a solid lesion; provide at least a first quantitative morphological parameter representative of the regularity of the outline of at least one object; and a second parameter representative of the content and/or consistency of an area of the ovarian, renal, pancreatic, bladder, gallbladder, hepatic solid lesion or cyst within the contour; processing the image using a second algorithm to estimate a benign or malignant classification of the cyst or solid lesion elaborating through a machine learning algorithm.

### CROSS REFERENCE TO THE RELATED APPLICATIONS

This application is the national phase entry of International Application No. PCT/IB2023/056227, filed on Jun. 15, 2023, which is based upon and claims priority to Italian Patent Application No. 102022000012623, filed on Jun. 15, 2022, the entire contents of which are incorporated herein by reference.

### TECHNICAL FIELD

The present invention refers to a processing method for an ultrasound image for the help of medical personnel in order to increase the repeatability of medical diagnoses and to reduce time and cost of managing the diagnostic procedure.

### BACKGROUND

The extraction of parameters useful for bladder and cyst tumour diagnosis, such as ovarian renal or pancreatic cysts, is deepened but the numerous parameters obtained must still require an important human intervention to be related to each other. Sometimes, the large amount of information can cause a situation wherein data potentially indicative of a pathology are present along with many others that tend to make the relevant data less recognizable. This poses a risk regarding the reliability of the diagnosis which thus becomes very dependent on the human factor e.g. from the experience of the practitioner who evaluates the data.

### SUMMARY

The scope of the present invention is to provide a selection of data extracted from ultrasound images in order to increase the reliability of the diagnosis and facilitate the interpretation by medical personnel by drawing the latter's attention to the most significant parameters of the ultrasound images. Furthermore, based on this selection, an estimate based on a group of histotypes is provided.

The scope of the present invention is achieved through a computerized method of processing an ultrasound image representative of an ovarian, renal, pancreas, bladder or cyst solid lesion, e.g. ovarian, renal, hepatic, pancreatic, bladder, gallbladder object of a subsequent medical diagnosis, comprising the steps of: segmenting the image through a first algorithm to: o identifying at least one contour and at least one corresponding object delimited by the contour and representative of a biological tissue of a cyst or a solid lesion; o providing at least:
a first quantitative morphological parameter representative of regularity of the contour of the at least one object and at least a further morphological quantitative parameter representative of the number of loculi inside the cyst;
a second parameter representative of the content and/or type of an area of the ovarian, renal, pancreas, bladder, gallbladder, liver cyst or solid lesion said second parameter comprising a localization of a serous zone or a spotting of a mucinous zone processing the photogram by means of a second algorithm to estimate a benign or malignant classification of the cyst or solid lesion processing through a machine learning algorithm the estimate of the benign/malign classification, the first second and the further parameters to estimate at least one or a group of histotypes congruent with the photogram.

The lesions object of the invention are tumors which on ultrasound examination are characterized by a common morphology, and which can therefore be described by comparable indicators; such as for example circular shape, presence of one or more loculi, presence of wall irregularities, as in the case of ovarian, renal, pancreatic, bladder masses with serous or partially serous content; and/or comparable texture indicators, such as ovarian, renal, pancreatic, hepatic, bladder, gallbladder, solid or partially solid masses.

Through the three phases indicated above, data for medical personnel are appropriately selected and processed to provide a compromise decision basis between the details, provided by the segmentation, and an overall assessment, provided by the classification. The estimate of a group of probable histotypes also provides medical personnel with an important indication regarding the subsequent conduct of the analyses: whether the histological examination is avoidable because e.g. the group of histotypes generated comprises all benign indications, medical personnel will also have at least the first and second quantitative parameters to perform a final evaluation which, if confirmed, will provide a considerable cost saving and increase in comfort for the patient.

Furthermore, having selected the essential parameters to provide a diagnosis, it is easier to perform a high degree of automation through image processing algorithms.

The congruence of the histotypes group is for example obtained through statistical analyses present in scientific literature wherein, at least the first and second quantitative parameters being provided and the benign/malignant indication with reference to a tumoral pathology, the histotypes more frequently associated to the input data are indicated in a group of at least two histotypes. Such operation is performed through an algorithm of machine learning with statistical inference or other less complex than an algorithm of deep learning based on neural networks.

For example, the identification of contours is based on the identification of connected components.

According to a preferred embodiment, the method comprises the step of receiving a region of interest and providing at least one of the first and second algorithm with a region of interest containing the ovarian, renal, pancreatic, bladder cyst or solid lesion.

In this way, it is possible to make the first and second algorithm more precise and faster since they are excluded from the analysis of pixels of poor interest.

According to a preferred embodiment, the method comprises the step of identifying through a third algorithm the ROI by photogram processing.

In this way it is possible to make the process entirely automatic.

According to a preferred embodiment, the method comprises the step of providing to the second algorithm the first and second quantitative parameters.

In this way, unless errors propagation, a particularly accurate final result is obtained.

According to a preferred embodiment, the benign/malignant category, the group of histotypes, the first quantitative parameter and the second parameter are reproduced to be visible to a user on the image outside the ROI.

In this way, the data is presented synoptically for the medical staff, therefore it is in the best conditions to make a diagnosis.

### BRIEF DESCRIPTION OF THE DRAWINGS

Preferred embodiments of the present invention will be described in the following description, purely byway of example, with reference to the attached drawings, wherein:

FIG. 1 shows a first embodiment of the method of the present invention;

FIGS. 2A-2C, 3A-3D, 4A-4D, 5A-5B, and 6A-6D show successive phases of a segmentation of a ultrasound photogram performed according to an exemplary embodiment of the present invention;

FIGS. 7-8 show corresponding block diagrams wherein segmentation and identification steps are respectively in parallel and in series with respect to a navigation phase to identify the ROI;

FIGS. 9A-9B, 10A-10C, 11A-11B, and 12A-12B show successive steps to obtain the ROI on the basis of an algorithm according to the present invention.

### DETAILED DESCRIPTION OF THE EMBODIMENTS

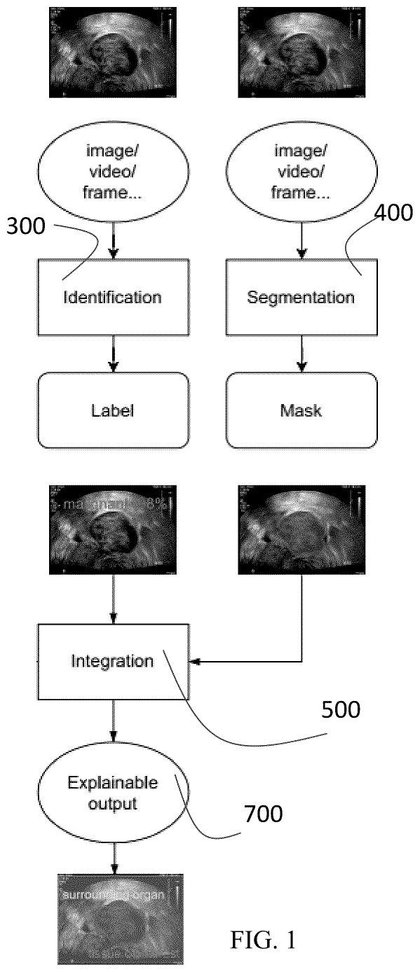

In FIG. 1 a first embodiment of the method of the present invention is shown. This embodiment provides a Segmentation step 300 and an Identification step 400 of an ultrasound photogram both single and from an ultrasound clip containing a putative cyst and/or a putative solid lesion of the ovary, kidney, pancreas or bladder.

Segmentation 300 aims to delimit and identify one or more image objects e.g. pixel areas, representative of an organ, such as an ovary, a bladder, a pancreas, a kidney and cyst or solid lesions on/in such organs. Segmentation 300 also provides quantitative morphological parameters based on the processing of radiomic parameters, i.e. on the texture and geometric information that can be obtained by analysing the various grey levels of the pixels of the photogram and relating these grey levels.

Examples of parameters representative of the consistence of cyst or solid lesion area are based on photogram texture analysis and allow to estimate whether an object e.g. an area of the photogram refers to a serous, mucinous, solid, vascularized, non-vascularized, papillary, mixed content area i.e. combination of serum, mucin and solid part.

Examples of quantitative morphological parameters are one or more of: the regularity of a closed edge of serous area of the cysts, the regularity of an external edge of a solid lesion, presence and number of loculi within the cyst, centroid of the cyst or solid lesion or loculi, characteristic area and/or size of the loculi, presence and number of solid excrescence and/or papillae, ratio between the area occupied by the solid and serous components of the cyst content, presence and area of non-solid hyperechoic regions (e.g. mucin or blood).

Step 300, which allows to identify the contour of an object and classify the contour and the object can be performed through different methods. This step allows to delimit the entire intended object from time to time, e.g. the cyst within the ROI rather than only portions of interest of the object, characterized by an uniform appearance inside them.

The input of this step is an ultrasound image, for example one or more photogram of an ultrasound clip, while the output is defined in the form of a contour of one or more objects, coordinates of each pixel included within the object. These in turn can be represented in the form of binary masks or probability masks or the value of a given parameter or a set of parameters, such as the range of values that can be assumed by the features automatically extracted from the trained neural network (value of the weights assumed by neurons according to a specific pattern for each label learned); the range of values that can be assumed by one/more radiomic features and associated with a specific learned label.

A first example of a processing algorithm involves the use of deep neural networks capable of performing semantic segmentation. Examples of architectures are:

UNet, Mask-R CNN, GCN. These networks typically have a two-stage structure. A first stage of encoder, which can be performed through a deep convolutional network, where the learning step takes place and the networks learn to recognize the object to be segmented. The second step is the decoder, where the parameters learned in the previous step are applied to the image to delimit the portion whose characteristics correspond to the characteristics learned. The output is generally an image of the same size as the image to be segmented where, however, each pixel results labelled as belonging or not to one or more objects to be segmented. This method does not require indications on which characteristics and/or parameters to be selected, but these are implicitly extracted from the encoder.

These methods involve a training step based on a set of images/photograms containing the object/objects to be segmented. The set of images is provided as input to the network, as well as the labels to be “learned”, applied manually or by other automatic segmentation systems, typically binary masks of the same size as the input image, where for the pixels corresponding to the object/objects have a non-zero value, while non-belonging pixels have a zero value.

The accuracy of the segmentation is measured by comparing the non-zero pixels in the input and output masks, through metrics that reward corresponding values, and penalize discordant values, for example IOU, Dice coefficient. In this case, for algorithm learning, images labelled by qualified medical personnel are used.

A second example of known technique involves the use of segmentation algorithms based on the morphology of the image and/or on the distribution of pixel values in the image. Examples of this group are the watershed method, binarization methods and the following method for detecting the contours of mixed content cysts i.e. serum, mucin and solid areas.

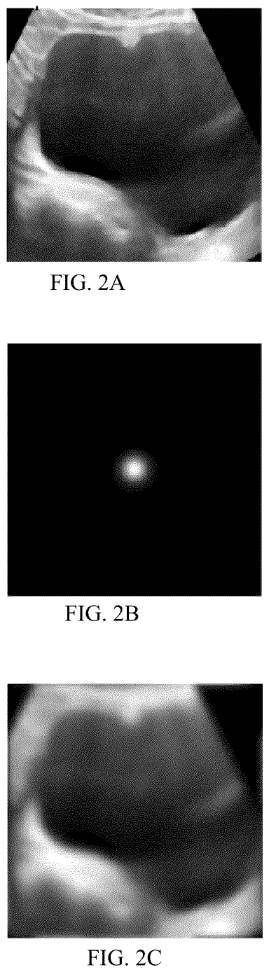

In FIGS. 2A-2C an ultrasound image of a scaled mucinous cyst e.g. at 300×300 pixels is shown, wherein in particular the mucinous part extends substantially over the entire area of the cyst. A mucinous area, as well as an area with blood effusions or relative to a fluid with a gelatinous or viscous consistence generating high scattering has a characteristic reduction of the contrast and, to be identified, the following steps are performed.

In order to increase the homogeneity or uniformity of the texture that makes up the region of interest, to be segmented, it is necessary to perform a smoothing operation, i.e. levelling. This homogeneity or uniformity can be defined as the distribution of the local standard deviation of the pixel value. Note that the reference parameter is the local standard deviation, i.e. calculated on a limited portion of adjacent pixels at time, not that total one, calculated on the entire image, which may be very high and does not vary even after levelling, for example due to the presence of an edge, even though it does not carry information about the dissimilarity of adjacent pixels.

A higher measure of local standard deviation indicates a greater variation in the value of the pixels under examination, and consequently a greater discrepancy of the texture, due to the scattering effect caused by the density of the liquid. A smaller measure of local standard deviation will indicate less difference and therefore more uniformity.

The application of a filter and the parameters that regulate the behaviour of said filter are, according to the invention, selected in order to obtain a significant reduction (in the enclosed examples a halving) of the average and maximum measure of local standard deviation calculated on the image original and filtered.

For example, in case of applying a frequency filter (FIGS. 2B-2C), the following is applied:
1. a Fourier transform to the image of FIG. 2A
2. a low-pass filter to image data resulting from the application of the transform, whose filter amplitude (hamming window) is preferably selected as the parameter that significantly reduces e.g. by 50% the local standard deviation of the region of interest (the area to be segmented) of the image in FIG. 2A. In case of cystic ultrasound images this value is about % of the input size (typically it varies between 50 and 100 pixels—300×300 pixel images)
3. Inverse Fourier transform.

It should be noted that for description purposes the images are illustrated with each of the transformations but, in use, the various steps are performed on a set of image data, each set being able to be converted and displayed if necessary in one of the images attached to the description. Alternatively, it is possible to process in frequency the ultrasound image through wavelet filtering, anisotropic diffusion or gaussian blur. For example, in case of anisotropic diffusion, three main parameters are important: kappa, controls the diffusion (smoothing) as a function of the image gradient. For low values of this parameter, very small gradients are sufficient to block the diffusion (typically it varies between 20 and 100 pixels-in the specific case the value of 90 was used). High values extend the diffusion even beyond high gradients (e.g. near high contrast contours); gamma: controls the rate of diffusion at each iteration (in our case=0.25); and iterations: number of repetitions of the algorithm on the same image, usually between 20 and 100 (in this specific case positive tests were performed with 30 iterations) depending on the non-homogeneity of the starting image (few iterations for homogeneous images, greater for images with high scattering).

Subsequently, the image is binarized e.g. isodata or through the Otsu algorithm, threshold identification with a two-component gaussian mixture model or generalized histogram thresholding.

In particular, with an isodated binarization, an algorithm automatically selects a threshold as the intermediate value between the average of the pixel values below the threshold and the average of the pixels above the threshold.

With reference to the Otsu binarization, an algorithm automatically selects a threshold as the value that minimizes the variance between pixel values within a class and maximizes the difference between classes.

In this way, two sub-sets of data are obtained respectively relating to areas considered representative of a low intensity or value of the pixel (or closer to the seed/inside the box) or a high intensity or value of the pixel (or distant from seed/outside the box).

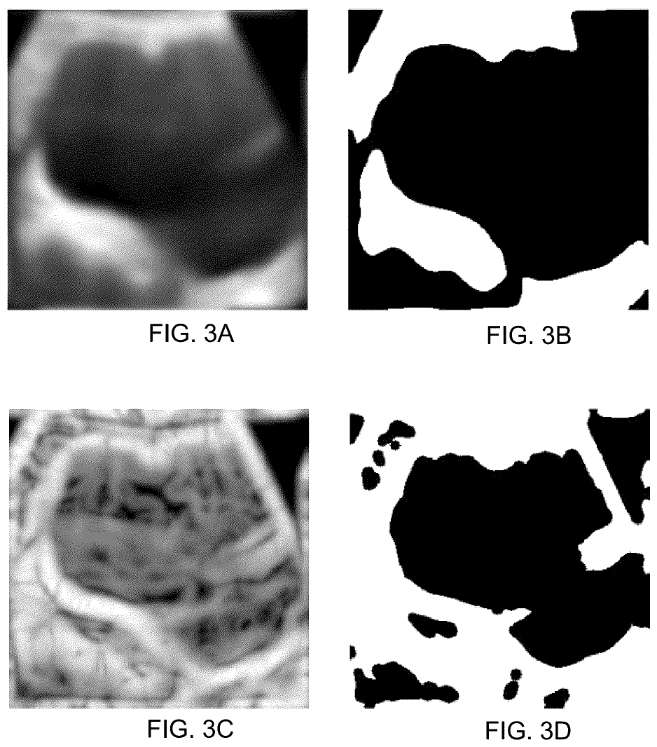

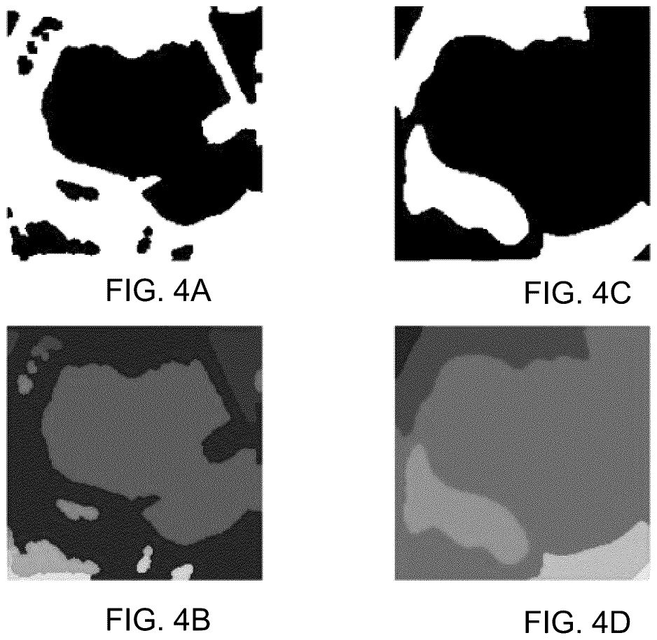

The ultrasound image of FIG. 2A is also processed to obtain information on homogeneity through an entropic filter (FIG. 3C). In particular, the information on homogeneity is represented by Shannon's entropy. On image of FIG. 3C a binarization algorithm is applied e.g. hysteresis thresholding. The latter performs a binarization on a variable threshold set as a parameter and the medium-high entropy threshold values, descriptive of the mucinous/blood effusion component. In particular, this binarization algorithm attributes the pixels of the input image to a class or to another on the basis of two selected thresholds. For each pixel, this is compared to the upper threshold. If the value is greater than the threshold, the pixel turns white and the surrounding pixels are compared with the lower threshold value, if they surpass it, it means that they are also white. This algorithm allows the identification of more uniform connected components and it is more resistant to noise and local variations versus binarization methods such as Otsu. In our example, the threshold that describes the characteristic distribution of entropy for mucinous areas is between 0.6 and 0.8 considering the local entropy of each pixel normalized between 0 and 1.

Alternatively, the image of FIG. 2A is processed through an algorithm of binarization of Li. In such case, the binarization algorithm receives a greyscale image as input, calculates a threshold based on cross entropy, so that pixels with value above the threshold belong to a first class (“figure”), those below to a second class (“background”). The threshold value is chosen in order to minimize the cross entropy between the pixel distribution of the figure and its average, and the pixel distribution of the background and their media.

In such step, a third and a fourth sub-set of image data are then generated relating to the area considered mucinous or highly scattered, for example through the attribution of the value 1, and the other categorized as a background or not belonging to the third sub-set, for example by assigning the value 0. It should be noted that an area with a high scattering e.g. due to the presence of mucin, it is always part of the cyst and never of a tissue area. Therefore, the third sub-set of data belongs to the same category as the first sub-set of data e.g. these are areas of the cyst and not of the background. This allows to segment the various sub-sets of data, with their related components, i.e. with reference to images binarized, connected regions with an area greater than 1 pixel composed by connected pixels, i.e. adjacent pixels that share the same value (or around the value, if it is coloured or grayscale images).

Preferably, image in FIG. 2C is also segmented to check the outermost contours of the cyst since processing through frequency filters is applied to decrease the relevance of the mucinous blood effusion components that may be, without such step, incorrectly classified as tissue areas. The integration of the two approaches, with the integration of fragmented segmented regions but well-defined extracted from the original filtered image, returns a correct approximation of the cystic area. For example, in case of region growing algorithm, a tolerance of 50 is set starting from the seed. The connected areas or components thus identified are subsequently segmented on the basis of the distance from the seed and grouped to obtain a fifth sub-set of image data representative of a distance of each area less than a maximum threshold and a sixth sub-set of image data representative of a distance of each area greater than said threshold. In this way, the fifth sub-set belongs to the first category of image data, e.g. the cyst area, and the sixth sub-set relates to the second category of image data, e.g. background.

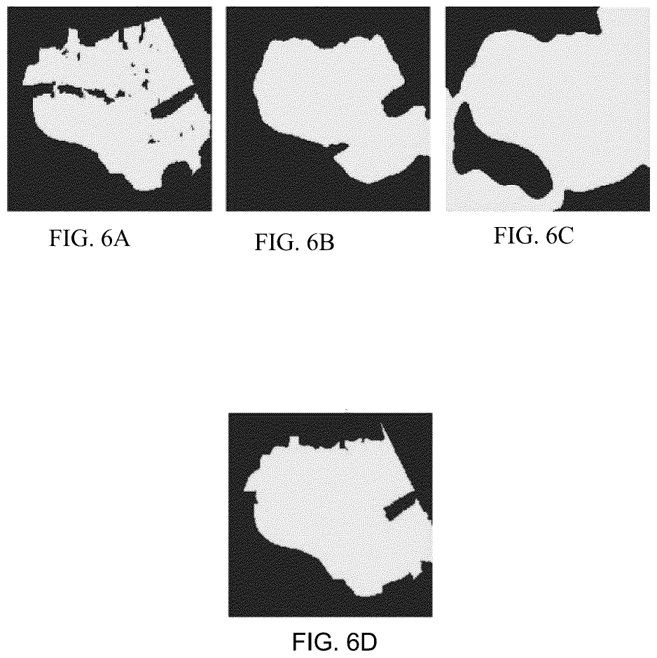

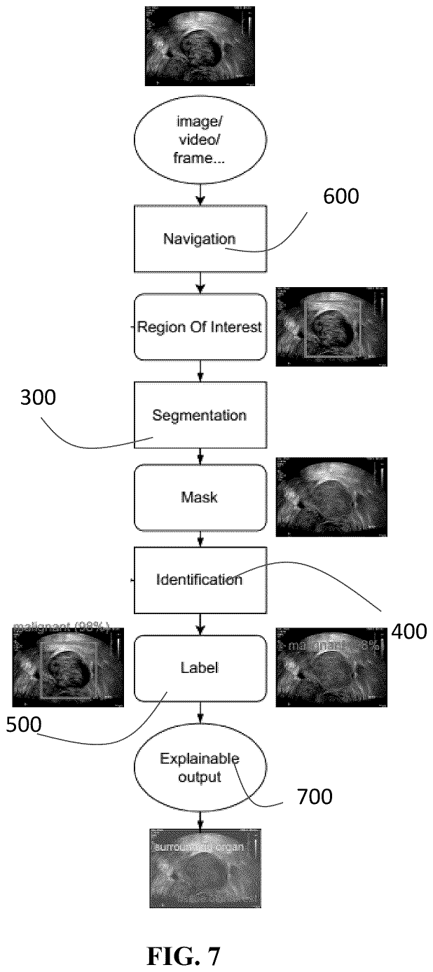

Subsequently, processing is performed on the basis of the sub-sets of image data so that to each value assigned after binarization e.g. 0 or 1, a meaning such as ‘background area’ or ‘cyst area’ has been applied through segmentation, and each subset of data includes at least the pixel value and its corresponding position. When it comes to sub-sets obtained by binarization, the value is e.g. 0 or 1, in case of the fifth and sixth sub-set of data, the areas are aggregated and a tag of the value 0 or 1 is assigned to each area of the same sub-set. For example, value 1 refers to all the sub-sets,-set of image data to areas with low intensity or close to the seed (bright colour in FIGS. 6A-6D) and value 0 is associated to a high intensity o distance from seed (dark colour in FIGS. 6A-6D). For example, the sub-sets of image data are combined based on the position of each pixel and the position results to be congruent for the three groups of data since the starting image e.g. FIG. 2A, is always the same.

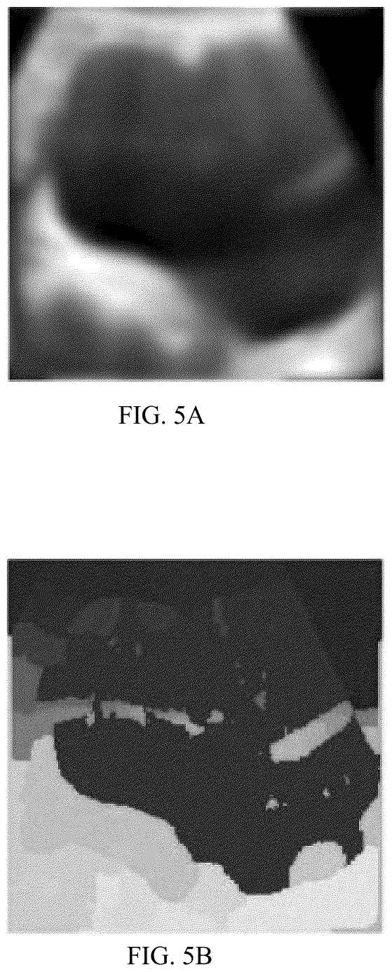

A result image can be generated so that, for each pixel position in the result image, the pixel value is the average of the values of the three pixels each belonging to the first and second data sets, and to the segmented data set (FIG. 5B and labelled). When the average is applied, the result image is displayed following the application of a new threshold to establish whether the pixel is of the first or second category.

Otherwise, it is possible to generate the result image by applying the mode of the three values of each pixel for a given position: each pixel acquires the mode value of the three values obtained from each of the three elaborations of FIGS. 6A-6C.

It is also possible to use correlation: cross correlation of binary masks in pairs, and average of correlated images. This last method returns a clear localization of the cystic area, but, in addition to computational power, it requires the selection of a threshold on the correlation value to select the range of pixels to be included in the final segmentation.

After identifying as above result image data (FIG. 10C) a cyst area, e.g. yellow area or with reference value 1, it is then possible to calculate the area, the centroid and other morphological parameters relating to the regularity of the edges of the binary mask.

According to an embodiment variant, the calculation of the local entropy is applied to the first set of data, e.g. to the result of the ‘smoothing’ phase in order to increase the effectiveness of identifying any areas with high scattering inside the cyst.

These algorithms do not require a training step, although the choice of parameters is made on the basis of the characteristics of the dataset to which the segmentation is applied. Assuming a set of test data, on which the area to be segmented manually or by other automatic segmentation systems has been delimited, the accuracy of the segmentation is measured by comparing the non-zero pixels in the original and in the output mask, through metrics that reward corresponding values, and penalize discordant values, for example: IOU, Dice coefficient.

A third example of implementation involves the use of algorithms based on the extraction of radiomic and textural features/parameters.

These methods, unlike the methods based on deep neural networks, require the calculation and subsequent classification of a set of characteristics, such as shape, texture, pixel value, etc., of the object to be segmented which are selected during the training step. A use of this method generally involves the steps of: calculating the characteristics selected from an entire object or from portions of images; dividing the calculated values between those belonging to the object or portion of the image with a certain identity and those with different identities; training a classifier, of machine learning type, neural network, etc., to associate the calculated values and the identities of the object/portion; sending as input to the trained classifier the new image and the assignment of an identity for each portion of the image. Portions with the same identity will be assigned the same label and can be represented through, for example, binary or coordinate masks. Assuming a set of test data, on which the area to be segmented manually or by other automatic segmentation has been previously delimited, the accuracy of the segmentation is measured by comparing the non-zero pixels in the original and the output mask, through metrics that reward corresponding values, and penalize discordant values (e.g.: IOU, Dice coefficient). A fourth example of known technique involves the use as a combination or subset of the previously described algorithms. The present invention also includes an Identification step 400 to preferably confirm the presence of the cyst or solid lesion and estimate a benign/malignant category of the cyst or solid lesion within the ultrasound photogram. For example, one or more of the photogram described above may have produced parameters relating to the contour and content such as to be able to hypothesize a cancerous lesion. Step 400 performs a photogram analysis in order to estimate a benign or malignant category to which the lesion belongs.

The identification step 400 can be performed by means of different methods and implementation techniques. For example, the input is an ultrasound image e.g. a photogram, an ultrasound video, which returns as output a benign or malignant category of belonging of the cyst or solid lesion and, preferably, the probability associated with the category to which it belongs.

A first example of implementation suitable for the scope involves the use of deep neural networks for the classification of the entire ultrasound image e.g. of the frame.

The architecture generally involves the use of a convolutional network (CNN). The input is a set of images representing objects, e.g. cysts or solid lesions, where each image is associated with a label that indicates a category or categories to which the object belongs. Scope of the learning step is to teach the algorithm the association between an image and the appropriate category, through the identification of parameters automatically extracted from the network from the training dataset, and which best represent a given category.

Neural network training takes place by dividing the available dataset into three subsets: a training subset, a validation subset, a test subset. Typically, the training subset is made up of 60% of the data, while the validation and test subsets are made up of the remaining 20 and 20%. Each subset shares the same properties, for example in terms of frequency of occurrence of a given label, origin of the data, characteristics of the data (size, appearance). The training consists in the learning by the network of a specific response pattern in association with the presentation of an input belonging to a given category. For effective classification, different categories will be associated with separate response patterns. The response pattern is made up of weights, i.e. the connections between the single neurons that make up the network. In a preferred version of the training, the network parameters are set as follows: learning rate (i.e. the coefficient of variation of the network weights at each iteration) is between 0.001 and 0.1; optimization algorithm (i.e. the approximation operation of the network weights pattern that maximize learning) is ADAM; the number of epochs (i.e. of iterations wherein the network is exposed to the training dataset and the weights are modified) is chosen adaptively through an early stopping algorithm, i.e. the iterations are interrupted, with a tolerance factor, when the prediction error on the training dataset decreases, but the one on the validation and test dataset increases (phenomenon called overfitting). The network parameters are selected iteratively by observing the performance obtained on the validation set. Once the selection process is completed, training is measured on the test dataset and the performance assessed. Learning is considered completed when the performances are greater than or equal to an established threshold (for example 90% of correct answers) and stable (i.e. with variations below a given threshold, for example plus or minus 3 percentage points) through reiterations of the process following variations in the combination of the sub-portions of the dataset.

In a preferred version of the invention, the dataset is subjected to operations of “Increase”, i.e. each data is randomly subjected to geometric operations (rotations, translations, scale) and to changes in appearance (contrasts and luminance), in order to increase the number and to limit the effect on the learning of random variables not replicable
(for example, all data collected in a medical centre have very low contrast values).

The output returned is a subset of the relevant images, together with the probability that the object belongs to the benign/malignant category with reference to a tumour pathology. The learning accuracy is calculated on a second set of images/photogram not present in the training dataset, with statistics and characteristics comparable to the training dataset, comparing the number of correctly categorized images, those missing, and false positives and obtaining accuracy, sensitivity, and specificity scores.

A second example of implementation involves the use of algorithms based on the extraction of characteristics, radiomic and textural parameters. These methods, unlike the methods based on deep neural networks, require the calculation and subsequent classification of a set of characteristics, such as shape, texture, pixel value, etc., of the object to be categorized which are selected by the experimenter in the training step. One use of this method involves: (1) calculation of the features selected from an entire object/image portions, (2) division between the calculated values between those belonging to the object/image portion with a certain identity and those with other identities, (3) training of a classifier (machine learning e.g. neural network, logistic regression, linear regression, decision trees, support vector machine, bayesian regression, perceptron, naive bayes in the case of supervised approaches; clustering algorithms such as nearest neighbours, k means, gaussian mixture in case of unsupervised approaches; and/or combinations of these to associate calculated values and identity of the object/portion, (4) administering a new image as input to the trained classifier and assigning an identity to the image.

Learning accuracy is calculated on a second set of images e.g. photograms not present in the training dataset, with statistics and characteristics comparable to the training dataset, comparing the number of images correctly categorized, missing, and false positives, and obtaining accuracy, sensitivity, and specificity scores.

A third example involves the use as a combination or subset of the previously described algorithms. Still considering FIG. 1, the outputs generated by steps 300 and 400 are processed in an estimation step of histotype 500 performed through a third machine learning algorithm to provide an estimate of a group comprising at least two histotypes congruent with the solid cyst or lesion. By histotype is meant the type of tumour as indicated by its cell characterization, i.e. the type of cells that form the tumour tissue, as detected by the microscope analysis of a tissue sample collected in a biopsy with reference to both cysts and lesions solid.

For example, there are 43 histotypes of ovarian cysts as indicated by references 1:17 in the bibliographic section. Each of these histotypes is also associated with a benign/malignant category, therefore knowing as input data the output of the identification step 400 allows to eliminate the histotypes incongruent with such data produced by the previous processing of the ultrasound image. It is also possible through relatively simple machine learning algorithms, e.g. excluding neural networks, set up a trained classification function that receives the outputs of steps 300 and 400 as input, possibly the congruent histotypes previously selected, and provides a group of at least two congruent histotypes and, preferably, the percentage that, in case histological examination performed with a cyst tissue or solid lesion, the outcome of such examination is in the estimated group.

Examples of machine learning algorithms that can be used are: logistic regression, linear regression, decision trees, support vector machine, bayesian regression, perceptron, naive bayes in the case of supervised approaches; clustering algorithms such as nearest neighbours, k means, gaussian mixture in the case of unsupervised approaches; and/or combinations thereof. The training of supervised models uses datasets equipped with labels indicating the histotype for each input data, in this case affixed by expert doctors. Through iterations, the algorithm selects the range of values of the parameters that define it that maximize learning, i.e. the model ability to assign the correct label to a given set of input data.

Learning defines the function underlying the model that best describes and separates the input data in case of discordant labels. The parameters that define the model are for example the slope and the intercept of the line in the case of linear regression, the polynomial in the case of the support vector machine, the depth or the number of branches generated in the case of a decision tree. It is also possible to define regularization terms, or terms added to the function that defines the model itself, and which assign a penalty to incorrect answers during learning. An example of regularization is the L2 for logistic regression. For some models such as support vector machine it is possible to define the function underlying the model, which can be linear, cubic, or an n dimensional polynomial.

The training of unsupervised models uses datasets without labels. The purpose of learning is to define a space (cluster) to which each input data belongs, in order to maximize the proximity of data belonging to the same space, and maximize the distance of different spaces. An example of a parameter that defines many clustering algorithms is the number of clusters allowed in output, that is, the number of categories that are implicitly detected and into which the incoming data can be divided.

Scope of the machine learning algorithms in defining the histotype is to associate a set of tumour input characteristics, such as morphological and textural characteristics, to the histotypic characterization of the tumour under examination.

For example, the algorithm is trained on the basis of real cases evaluated by experienced and reputable medical personnel on the basis of the same inputs provided by the segmentation step 300 and identification 400 and subsequently object to histological analysis.

According to a preferred embodiment, the method of the present invention comprises a navigation step 600 to produce a region of interest (RO I) containing a cyst or solid lesion in the ultrasound image. Such step may involve receiving manually generated data, e.g. the medical personnel independently indicates the RO I, or, preferably, an algorithm is set up to process the ultrasound image and automatically generate the ROI. The latter embodiment is particularly useful when the input is an ultrasound clip: it is in fact possible that some photograms do not include cysts or solid lesions.

By region of interest, or ROI, it is meant a portion of the ultrasound image delimited by a visual indicator within which is located the cyst or solid lesion on which performing detections, measurement and/or data analysis. The indicator can be of different shapes such as polygonal, circular, elliptical; the most commonly adopted shape is the rectangular one. Preferably, the navigation step 600 also returns an indication of the absence of a ROI in the event that an area containing a cyst or a solid lesion cannot be identified.

ROI allows to reduce observational and computational efforts to a specific area, leaving out the remaining part of the ultrasound image and increasing processing speed.

The navigation step 600 has an input represented by an ultrasound image, e.g. a photogram or from an ultrasound video and the returned output includes: o a subset of the ultrasound images on each of which the delimitation of the region of interest is applied; o a delimitation by means of a bounding box of a sub-area of the image or photogram within which the ROI is contained; o a ROI localization, by localization of the centroid of the region A first example of a step performed automatically to obtain a subset of ultrasound images containing a ROI is through the use of deep neural networks to perform a classification.

The architecture is called a convolutional network (CNN), and is trained on a set of images/photograms that contain or do not contain a region of interest. The learning accuracy is calculated on the basis of a second set of images/photograms that was not used in the training step, but with statistics and characteristics comparable to the training dataset, as previously indicated in the segmentation step 300. Comparing the number of regions of interest identified, those not identified and the false positives results in accuracy, sensitivity, and specificity scores. A second example of a step performed automatically to obtain a delimitation through a bounding box of the area occupied by the ROI is through the use of neural networks of the object detection type.

The training in this case requires the input to be a set of images with the corresponding bounding box coordinates around the object to be identified. These networks analyse sub-portions of the image and, for each, extract and classify characteristics of n degree automatically extracted from the neural network, they are outputs of the mathematical operations of the various neurons in the network, and of the operations of combining the output of the neurons that occur to each layer of the network starting from the input, inputs which are the ordered pixel values of the image. These are compared with those contained in the sub-portion indicated as the image label in the training step. The algorithm learns to recognize the combination of features that maximizes the probability of identifying the desired object and consequently its position. Also, in this case the learning accuracy is calculated on a second set of images/photograms that was not used in the training step, but with statistics and characteristics comparable to the dataset used in the training step.

The boxes affixed by the algorithm are compared in terms of overlap and size with those affixed manually. The most commonly used metrics for evaluating the goodness of the boxes are the intersection over union (IOU) and mean average precision applied to the bounding boxes, the real area and position (affixed by the user) and predicted (by the network) of the bounding boxes are compared. A third example of a step performed automatically to obtain a binarized map if the known parameters are subjected to thresholding, bounding box, or the centroid of the isolated portion and is obtained by means of segmentation algorithms based on the morphology of the image and/or on the distribution of the pixel values in the image.

If the object of interest has known properties, such as for a cyst: the presence of serum, the presence of highly echogenic edges, the presence of hyperechoic areas, these are searched for within the image and the portions of the image that more approximate these properties are delimited. In case the object is not present in the image, the algorithm does not identify portions that match selection criteria. After identifying the area of the image as above, a magnification factor, for example of 1.5, is applied to increase the probability that the area thus defined includes all the morphological characteristics and the corresponding pixels of the cyst or of the solid lesion.

FIG. 7 shows a further embodiment of the invention.

In this embodiment to the ultrasound image that can possibly be extracted from an ultrasound clip the Navigation step 600 is applied. The navigation step, as described above, has the scope of detecting a region of interest within the ultrasound photogram and representing an organ, a cyst or a solid lesion.

The output of this step is the identification within the ultrasound image of the image portion wherein the region of interest is contained delimited by a bounding box wherein the object of interest is present or, e.g. in case of a photogram, the indication that there are no cysts or solid lesions, so that the next photogram can be processed.

The output of the Navigation step 600 becomes the input for the subsequent steps of Segmentation 300 and Identification 400, in other words these two steps will be applied only to the portion of the ultrasound image identified by the ROI.

The Segmentation step 300 therefore, applied to the ROI only, classifies the object contained within the ROI on the basis of the biological tissue and the regularity of the contour of that object. The output of this step is one or more binary masks each of which identifies a portion of the object having certain characteristics.

The output of the previous Segmentation step 300, in this preferred form, becomes the input for the subsequent Identification step 400 which has the task of estimating the belonging to the benign/malignant category of the at least one object present within the ROI and of which parameters have been identified e.g. relating to the regularity of the contour and the variety of the content of the pixel areas delimited by the contours. Such parameters contribute to the processing to obtain the estimate of the benign/malignant category.

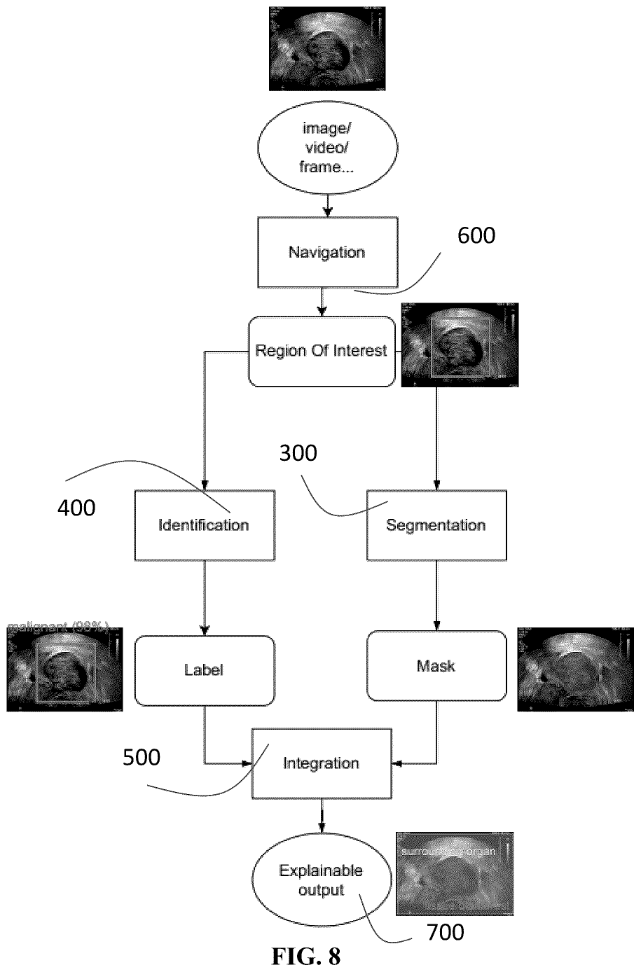

FIG. 8 illustrates a variant wherein the Segmentation step 300 and the Identification step 400 are in parallel with each other with respect to the navigation step 600, being the input ROI for each of the steps 300 and 400. In this way, it is possible to avoid the possible propagation of errors in one of the steps 300 and 400.

According to a preferred embodiment, the method further comprises the step of applying 700 to the processed image, i.e. the image initially processed in the Identification step 600, the results of the elaborations with a graphic readable by the medical personnel when e.g. displayed on a display of an ultrasound device or connected to an ultrasound machine, preferably outside the ROI and/or in an area of the image labelled during the segmentation step 300 as a background.

According to a preferred embodiment, when the results of the Segmentation step 300 and Navigation step 600 are obtained in parallel, the method comprises a congruence check step, preferably to check whether the ROI and one or more objects or contours identified during the step of Segmentation 300 are at least partially overlapped. Also such data, preferably, is then made accessible to medical personnel.

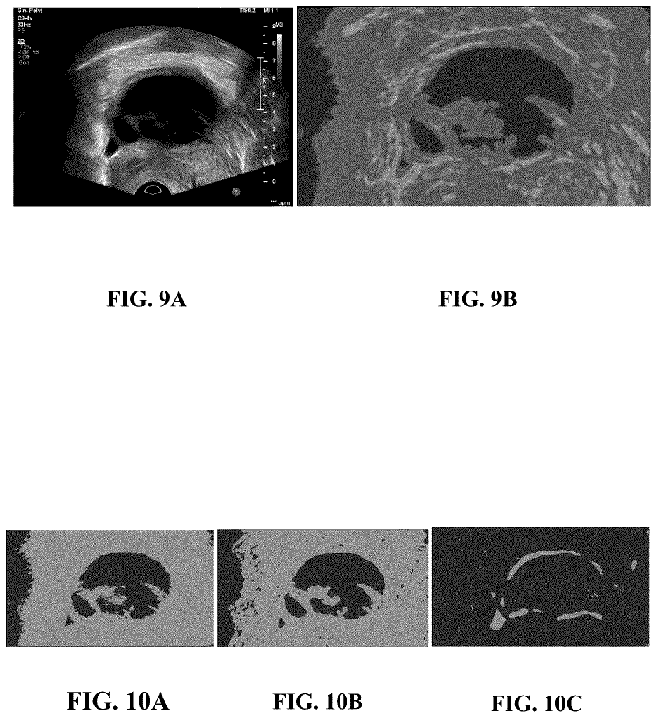

FIGS. 9A-9B, 10A-10C, 11A-11B, and 12A-12B illustrate an embodiment of a mixed algorithm to implement the navigation step 600. For example, the entropy of the image is calculated using an entropic filter (FIGS. 9A-9B) in order to make the areas whose grey levels are homogeneous more evident. Indicatively, this makes both the serous areas and the surrounding tissues more visible, when actually visually homogeneous. Alternatively, it is possible to apply filters to Hessian matrices.

Subsequently, the crop of the entropic image is object to binarization through special thresholds and then segmented in order to extract three different segmentations. Two of these (FIGS. 9A-9B) represent an approximation of the serous area with respective different thresholds but both low to identify the areas with high contrast and low entropy, the remaining instead the contour tissues (FIG. 10C) through the application of a high threshold, i.e. to identify areas with high entropy. The two approximations of the serous area, therefore internal to the cyst, have slightly different visual characteristics and are useful for validating the results. In particular, the two internal segmentations differ in that the respective thresholds adopted for binarization have similar but not identical values.

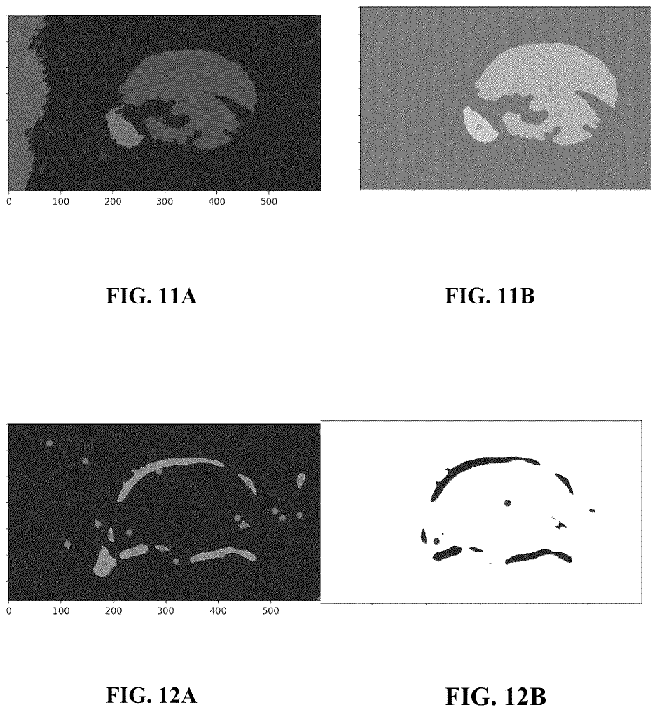

Specifically, taking the two segmentations from the inside, the centroids of the connected dark components are calculated, i.e. the closed areas representative of the serous areas, of an image (FIG. 11A) and it is verified that they are contained within the connected components of the other segmentation with a low threshold. When this and other conditions are verified, e.g. area of the connected component above a predetermined and settable threshold, distant connected component, e.g. outside a predetermined and settable limit distance, from the edges of the ultrasound, and a predetermined and settable form factor on the basis of the ratio between a major axis and a minor axis of the connected component approximated by an ellipse, then the component is considered connected as effectively representative of a serous area (FIG. 11B).

In this way it is possible to locate any number of serous areas, the maximum number of which is defined by means of a specific modifiable parameter; consequently, the method of the present invention is capable of identifying multilocular cysts and locating their individual loculi. The identification of the loculi with the corresponding position comes subsequently used to define the extension on the image of the RO I, i.e. the ROI must contain all the loculi.

The segmentation from the outside, i.e. the one that identifies the contours of the cyst, must be compared with the serous areas actually identified and selected in the previous step. Preferably, only the contours sufficiently close to the serum are selected.

To calculate the distance between the contour tissues and the serous area, the Euclidean distances between the centroids of the serous zones (FIG. 11B) and those of the single segmented contours are considered (FIG. 12A): if such distance is less than a predefined and settable threshold, the contour under examination is associated with the area and therefore selected (FIG. 12B). The predefined threshold can be fixed or adaptive, i.e. be expressed as a function of the size of the serous area, for example of one or both of the half axes. In this way to each loculi its contours will be associated.

Once both the serous areas and the relative contours have been extracted, ROI is identified, e.g. a box is placed around this area, which has as its side the maximum length and maximum height covered by the mask, with a possible scale factor to include any undetected portions.

### LITERATURE

[1] D. Timmerman et al. “ESGO/ISUOG/IOTA/ESGE Consensus Statement on preoperative diagnosis of ovarian tumors”, en. In: Ultrasound in Obstetrics& Gynecology 58.1 (lug. 2021), pp. 148-168. issn: 0960-7692, 1469-0705. doi: 10.1002/uog.23635. url:
[2] Francesca Moro et al. “Ultrasound evaluation of ovarian masses and assessment of the extension of ovarian malignancy”, en. In: The British Journal of Radiology 94.1125 (set. 2021), p. 20201375. issn: 0007-1285, 1748-880X. doi: 10.1259/bjr.20201375. url:
[3] A. C. Testa et al. “Imaging in gynecological disease (1): ultrasound features of metastases in the ovaries differ depending on the origin of the primary tumor”, en. In: Ultrasound in Obstetrics and Gynecology 29.5 (mag. 2007), pp. 505-511. issn: 09607692, 14690705. doi: 10.1002/uog.4020. url:
[4] V. N. Demidov et al. “Imaging of gynecological disease (2): clinical and ultrasound characteristics of Sertoli cell tumors, Sertoli—Leydig cell tumors and Leydig cell tumors”, en. In: Ultrasound in Obstetrics and Gynecology 31.1 (gen. 2008), pp. 85-91. issn: 09607692, 14690705. doi: 10.1002/uog.5227.
[5] C. Van Holsbeke et al. “Imaging of gynecological disease (3): clinical and ultrasound characteristics of granulosa cell tumors of the ovary”, en. In: Ultrasound in Obstetrics and Gynecology 31.4 (apr. 2008), pp. 450-156. issn: 09607692, 14690705. doi: 10.1002/uog.5279.
[6] L. Savelli et al. “Imaging of gynecological disease (4): clinical and ultrasound characteristics of struma ovarii”, en. In: Ultrasound in Obstetrics and Gynecology 32.2 (ago. 2008), pp. 210-219. issn: 09607692, 14690705. doi: 10.1002/uog.5396. url:
[7] D. Paladini et al. “Imaging in gynecological disease (5): clinical and ultrasound characteristics in fibroma and fibrothecoma of the ovary”, en. In: Ultrasound in Obstetrics and Gynecology 34.2 (ago. 2009), pp. 188-195. issn: 09607692, 14690705. doi: 10.1002/uog.6394.
[8] S. Guerriero et al. “Imaging of gynecological disease (6): clinical and ultrasound characteristics of ovarian dys germinoma”, en. In: Ultrasound in Obstetrics & Gynecology 37.5 (mag. 2011), pp. 596-602. issn: 09607692. doi: 10.1002/uog.8958. url: https://onlinelibrary.wiley.com/doi/10.1002/uog.8958.
[9] I. Dierickx et al. “Imaging in gynecological disease (7): clinical and ultrasound features of Brenner tumors of the ovary: Brenner tumors of the ovary”, en. In: Ultrasound in Obstetrics & Gynecology 40.6 (die. 2012), pp. 706-713. issn: 09607692. doi: 10.1002/uog.lll49. url:
[10] D. Franchi et al. “Imaging in gynecological disease (8): ultrasound characteristics of recurrent borderline ovarian tumors: Borderline ovarian tumor recurrence”, en. In: Ultrasound in Obstetrics & Gynecology 41.4 (apr. 2013), pp. 452-158. issn: 09607692. doi: 10. 1002 I uog. 12276. url: https: I/onlinelibrary.wiley.com/doi/10.1002/uog.12276.
[11] F. Moro et al. “Imaging in gynecological disease (11): clinical and ultrasound features of mucinous ovarian tumors: Clinical and US characteristics of mucinous ovarian tumors”, en. In: Ultrasound in Obstetrics & Gynecology 50.2 (ago. 2017), pp. 261-270. issn: 09607692. doi: 10.1002/uog.17222.
[12] F. Moro et al. “Imaging in gynecological disease (12): clinical and ultrasound features of invasive and non-invasive malignant serous ovarian tumors”, en. In: Ultrasound in Obstetrics & Gynecology 50.6 (die. 2017), pp. 788-799. issn: 09607692. doi: 10.1002/uog.17414. url: https://onlinelibrary.wiley.com/doi/10.1002/uog.17414
[13] F. Moro et al. “Imaging in gynecological disease (13): clinical and ultrasound characteristics of endometrioid ovarian cancer: Endometrioid ovarian cancer”, en. In: Ultrasound in Obstetrics & Gynecology 52.4 (ott. 2018), pp. 535-543. issn: 09607692. doi: 10.1002/uog.19026
[14] F. Pozzati et al. “Imaging in gynecological disease (14): clinical and ultrasound characteristics of ovarian clear cell carcinoma”, en. In: Ultrasound in Obstetrics & Gynecology 52.6 (die. 2018), pp. 792-800. issn: 0960-7692, 1469-0705. doi: 10.1002/uog.19171.
[15] B. A. Virgilio et al. “Imaging in gynecological disease (16): clinical and ultrasound characteristics of serous cystadenofibromas in adnexa”, en. In: Ultrasound in Obstetrics & Gynecology 54.6 (die. 2019), pp. 823-830. issn: 0960-7692, 1469-0705. doi: 10.1002/uog.20277.
[16] P. Anfelter et al. “Imaging in gynecological disease (17): ultrasound features of malignant ovarian yolk sac tumors (endodermal sinus tumors)”, en. In: Ultrasound in Obstetrics & Gynecology 56.2 (ago. 2020), pp. 276-284. issn: 0960-7692, 1469-0705. doi: 10.1002/uog.22002
[17] F. Moro et al. “Imaging in gynecological disease (22): clinical and ultrasound characteristics of ovarian embryonal carcinomas, non-gestational choriocarcinomas and malignant mixed germ cell tumors”, en. In: Ultrasound in Obstetrics & Gynecology 57.6 (giu. 2021), pp. 987-994. issn: 0960-7692, 1469-0705. doi: 10.1002/uog.23529. url:

What is claimed is:

1. A computer implemented method of processing an ultrasound image representative of an ovarian, renal, pancreas, bladder, or cyst solid lesion, comprising ovarian, renal, hepatic, pancreatic, bladder, and gallbladder object of a subsequent medical diagnosis, wherein the computer implemented method comprises steps of:
segmenting the ultrasound image through a first algorithm to:
identify at least one contour and at least one object delimited by the at least one contour and representative of a biological tissue of a cyst or a solid lesion;
provide at least:
a first quantitative morphological parameter representative of a regularity of the at least one contour of the at least one object and at least a further quantitative morphologic parameter representative of a number of loculi inside the cyst;
a second parameter representative of a content and/or consistence of an area of ovarian, renal, pancreas, bladder, gallbladder, liver cyst or solid lesion within the at least one contour, wherein the second parameter comprises a localization of a serous zone or a spotting of a mucinous zone;
processing the ultrasound image by means of a second algorithm to estimate a benign or malignant classification of the cyst or the solid lesion;
processing through a machine learning algorithm to estimate benign/malign classification, the first quantitative morphological parameter, the second parameter, and the further quantitative morphologic parameter to estimate at least one or a group of histotypes congruent with a frame.

2. The computer implemented method according to claim 1, further comprising a step of receiving a region of interest of the ultrasound image containing the cyst or the solid lesion to provide the region of interest in input to at least one of the first algorithm and the second algorithm.

3. The computer implemented method according to claim 2, further comprising a step of identifying the region of interest through a third algorithm, the step of identifying being previous to the step of receiving.

4. The computer implemented method according to claim 1, further comprising providing the second algorithm with the first quantitative morphological parameter and the second parameter.

5. The computer implemented method according to claim 1, further comprising applying on the ultrasound image outside a region of interest or in an image area labelled as background during the step of segmenting.

6. The computer implemented method according to claim 1, wherein the step of segmenting is a semantic segmentation step.

7. (canceled)

8. The computer implemented method according to claim 1, wherein the ultrasound image is an ultrasound frame.

9. The computer implemented method according to claim 2, further comprising providing the second algorithm with the first quantitative morphological parameter and the second parameter.

10. The computer implemented method according to claim 3, further comprising providing the second algorithm with the first quantitative morphological parameter and the second parameter.

11. The computer implemented method according to claim 2, further comprising applying on the ultrasound image outside the region of interest or in an image area labelled as background during the step of segmenting.

12. The computer implemented method according to claim 3, further comprising applying on the ultrasound image outside the region of interest or in an image area labelled as background during the step of segmenting.

13. The computer implemented method according to claim 4, further comprising applying on the ultrasound image outside a region of interest or in an image area labelled as background during the step of segmenting.

14. The computer implemented method according to claim 2, wherein the step of segmenting is a semantic segmentation step.

15. The computer implemented method according to claim 3, wherein the step of segmenting is a semantic segmentation step.

16. The computer implemented method according to claim 4, wherein the step of segmenting is a semantic segmentation step.

17. The computer implemented method according to claim 5, wherein the step of segmenting is a semantic segmentation step.

18. The computer implemented method according to claim 2, wherein the ultrasound image is an ultrasound frame.

19. The computer implemented method according to claim 3, wherein the ultrasound image is an ultrasound frame.

20. The computer implemented method according to claim 4, wherein the ultrasound image is an ultrasound frame.

21. The computer implemented method according to claim 5, wherein the ultrasound image is an ultrasound frame.
